(19) United States
(12) Patent Application Publication
(10) Pub. No.: US 2024/0197258 A1
(43) Pub. Date: Jun. 20, 2024

(54) Positioning a Patient Couch in a Magnetic Resonance System

(71) Applicant: Siemens Healthcare GmbH, Erlangen (DE)

(72) Inventor: Stephan Biber, Erlangen (DE)

(73) Assignee: Siemens Healthcare GmbH, Erlangen (DE)

(21) Appl. No.: 18/524,278

(22) Filed: Nov. 30, 2023

(30) Foreign Application Priority Data
Dec. 20, 2022 (EP) 22214872.8

(51) Int. Cl. A61B 5/00; A61B 5/055; G01R 33/30

(52) U.S. Cl. CPC A61B 5/704; A61B 5/055; G01R 33/307

(57) ABSTRACT

Method for positioning a couch in an MR system tunnel, including: positioning a position determination unit having a magnetic field sensor at a marker position on the couch or a patient supported on the couch; evaluating sensor data of a magnetic field sensor using a control unit operable to establish the marker position in a coordinate system of the MR system, in which a FOV position of a FOV of the MR system is known; determining a desired required position of the FOV relative to the marker position for a subsequent measurement of MR data in a direction of movement of the couch corresponding to the longitudinal direction of the couch by the control unit from the marker position; and activating the couch by the control unit using the marker position and the FOV position such that the desired position of the FOV relative to the marker position is set.

### TECHNICAL FIELD

The disclosure relates to a method for positioning a patient couch in a patient tunnel of a magnetic resonance system, wherein the, in particular, cylindrical patient tunnel is defined by a main magnet unit with a main magnet generating a main magnetic field. As well as this, the disclosure relates to a position determination unit and to a magnetic resonance system.

### BACKGROUND

In magnetic resonance imaging, nuclear spins of an object to be examined, in particular, of a patient, are aligned by a main magnetic field (B0 field), are excited, and the decay of the excitation is measured. In this case, the field of view of a magnetic resonance system is defined by the so-called homogeneity volume in which the homogeneity of the main magnetic field satisfies specific requirements, which allow high-quality magnetic resonance imaging. Today's magnetic resonance systems usually have a main magnet unit with a superconducting main magnet, which define a cylindrical patient tunnel into which the patient can be moved by means of a patient couch. In order to obtain a good imaging result, it is important to position the examination region of the patient as accurately as possible in the field of view of the magnetic resonance system. This is usually located in the hard-to-access cylindrical patient tunnel. Due to this constellation, the middle of the field of view in a magnetic resonance system is partly also referred to as the isocenter of the magnetic resonance system and can coincide with the middle of the patient tunnel.

In modern magnetic resonance systems, it should be possible for a user to position the examination region in as simple as possible a manner, in particular, while simplifying the workflow, also taking into account where necessary local coils/local coil arrangements to be used, in the field of view of the magnetic resonance system. Solutions have already been proposed for this in order to define target positions when the patient couch has not yet been moved into the patient tunnel, in particular, in the longitudinal direction of the patient couch, which also corresponds to its direction of movement into the patient tunnel and, with a cylindrical patient tunnel, usually also corresponds to the longitudinal direction of the cylindrical patient tunnel. This direction is frequently referred to as the z-direction, the horizontal direction at right angles to this as the x-direction, and the vertical direction at right angles to the z-direction as the y-direction.

To determine a longitudinal position (z-position) of the patient couch, which is to be positioned in the field of view, in particular, in the middle of the field of view, it has been proposed in this case that, for example, a laser marking unit be provided above the entry into the patient tunnel, which projects a marking point or a marking line from above onto the patient couch (or onto the patient/local coil arrangements on the patient). With this, in particular, a position on the patient can be determined that the operating personnel can verify visually. Since the couch position and the position of the field of view are known to a control system of the magnetic resonance system, a corresponding offset to the field of view can be determined, by which the patient couch will be moved in order to move the patient into the patient tunnel in such a way that the position marked with the laser marking system matches the middle of the field of view, at least in the longitudinal direction. However, the operation of the laser marking system has, above all, proved difficult to the extent that the patient must ultimately be placed by moving the patient couch suitably in relation to the marking, and then a confirmation must still take place.

In another approach, it has been proposed that markings and/or operating unit be provided along the edge of the patient couch, so that, by entering the corresponding marking or by actuating the corresponding operating unit, likewise a longitudinal position along the patient couch can be selected, which is to be positioned in the field of view, in particular, the middle of the field of view. However, only an extremely coarse positioning specification is possible with this. This is at odds with highly-accurate operating options of motors in magnetic fields. For example, in DE 10 2020 211 327 A1, a couch with a DC motor is proposed, in which, by means of a Hall sensor, for example, an angle setting of windings of the motor in relation to the main magnetic field is to be measured in order to select a suitable winding. DE 10 2017 202 399 A1 describes a method and an apparatus for positioning in a magnetic field of a magnetic resonance tomograph, wherein the couch can have a plurality of magnetic field strength sensors, which are provided spaced out in its longitudinal direction or direction of movement.

DE 10 2016 203 255 A1 discloses a method and an apparatus for position determination in a magnetic resonance tomograph, in which it is proposed that an extremely accurate z-reference be determined when moving the mobile apparatus into the patient tunnel, in that a so-called sweet spot is detected, at which, for a plurality of pairs of x-y coordinates, the magnetic field strength assumes essentially the same value for an equal z-coordinate value. Therefore, a magnetic field strength sensor is arranged in the mobile apparatus, with the aid of which a check is made as to whether the characteristic magnetic field strength is reached at this point. In this way, a reference value for the z-coordinate can be defined in a simple way, and subsequent movements can be related thereto.

Independent of the grammatical term usage, individuals with male, female, or other gender identities are included within the term.

### SUMMARY

An underlying object of the disclosure is to specify a method and an aid for definition of a target position for the patient couch, at least in its longitudinal direction, that simplifies workflows yet is still sufficiently accurate.

To achieve this object, in a method of the type mentioned at the outset, there is inventive provision
for a position determination unit comprising an, in particular, three-dimensional magnetic field sensor to be positioned on the patient couch or on a patient supported on the patient couch at a marker position,
for the sensor data of the magnetic field sensor for determining the marker position in a coordinate system of the magnetic resonance system, in which a field of view position of a field of view of the magnetic resonance system is also known, to be evaluated by means of a control system,
for, at least in a longitudinal direction corresponding to a direction of movement of the patient couch, a desired required position of the field of view relative to the marker position to be determined for a subsequent measurement of magnetic resonance data by the control system from the marker position, and
for the patient couch to be activated in such a way by the control system, using the marker position and the field of view position, that the desired relative required position of the field of view is set in relation to the marker position.

The longitudinal direction of the patient couch and thus a direction of movement in which the patient couch is able to be moved can, in particular, correspond here to the longitudinal direction of the, in particular, cylindrical patient tunnel, can thus represent the z-direction of the magnetic resonance system often referred to in this way. The horizontal direction at right angles thereto can be seen as the x-direction, the vertical direction at right angles to the z-direction as the y-direction. In a coordinate system of the magnetic resonance system defined in such a way, for example, the field of view position is usually known. It can be conceivable, for example, to set the middle of the field of view as the origin of the coordinate system, which especially advantageously can correspond to the middle of the patient tunnel. In this coordinate system, a current couch position of the patient couch can also be determined. In accordance with the disclosure, it is now proposed to use an, in particular, compact, easy-to-handle position determination unit, which is capable, on the one hand, of optically describing a selected marker position for an operator through its arrangement, but on the other hand allows, by means of a magnetic field sensor provided therein, in particular, a magnetic field strength sensor, this marker position to be sufficiently accurately defined at least in the z-direction and at least outside the patient tunnel, thus, in particular, in the stray field of the main magnetic field, at least in the longitudinal direction in the coordinate system of the magnetic resonance system.

To do this the magnetic field sensor thus measures the magnetic field strength and does so, in particular, as a three-dimensional magnetic field vector, wherein it can also be conceivable to measure an amount of three-dimensional magnetic field vector as magnetic field strength. If a main magnetic field map, which at least describes the stray field outside the patient tunnel, or thus a stray field map, is present due to the measured magnetic field strength at least in the z-direction, the position of the magnetic field sensor can also be determined. The main magnetic field map, describing the stray field outside the patient tunnel, is available here in the coordinate system of the magnetic resonance system so that, by reconciling the sensor data of the magnetic field sensor with the main magnetic field map, a highly accurate position determination is possible, in particular, when the three-dimensional magnetic field vector is supplied, thus also a direction of the measured magnetic field.

Thus, if the position determination unit has been positioned by visual assessment by an operator, for example, the sensor data of the magnetic field sensor can be evaluated, in particular, using a main magnetic field map, in order to also have available a corresponding marker position electronically in the coordinate system of the magnetic resonance system. Moreover, it is known to the control system how the field of view is to be positioned in relation to the marker position, in particular, at least in the longitudinal direction (z-direction), the marker position is to be moved into the middle of the field of view, a movement path of the patient couch in the longitudinal direction can be determined in a simple manner in order to produce the desired required position of the field of view relative to the magnetic field sensor, i.e., the marker position, of the position determination unit, by controlling the patient couch accordingly. The position determination unit is thus to be understood as a clever combination of optical marker and localizable electronic markers and can, therefore, also be referred to as a position tag or position marker, respectively.

In this case, use is made of the fact that the patient on the patient couch is usually prepared outside the patient tunnel for the measurement and also the definition of the measurement positions for magnetic resonance data, which the patient couch is to adopt, usually takes place in the workflow in this preparation phase. In this case, the patient couch with the patient is located, depending on position, in the widely differing, i.e., spatially varying, stray field of the main magnetic field, which can be measured and taken as the basis for a precise position determination by means of the magnetic field sensor, at least while the patient couch with the patient is located in just this stray field.

The position determination unit, in this case, is preferably embodied as a small, easy-to-manage apparatus, which preferably does not serve any purpose during the subsequent measurement of the magnetic resonance data and, in particular, does not disrupt the measurement. In particular, the position determination unit can be embodied separately to local coil arrangements to be used for measurement of magnetic resonance data or other, larger aids for measurement, in order in the simplest possible way to be able to indicate the position optically and in doing this, be independent of other aids, where necessary having specific positioning requirements or restricted in their positioning.

In other words, the position determination unit can thus be understood as a position tag or position marker, respectively. The position determination unit is positioned at the marker position on the patient couch (or on the patient arranged thereon), which is to be moved by the magnetic resonance system into the field of view or of which the location in relation to the field of view is to be clearly defined. The magnetic resonance system recognizes by means of the control system the marker position of the position determination unit and then knows the required position and the wish of the user expressed therewith for positioning of the patient by means of the patient couch with a corresponding target coordinate relative to the field of view. Thus, a simple-to-manufacture, small and versatile, preferably wireless, position determination unit is produced, which, by positioning it on the patient couch or the patient, can be used to notify the magnetic resonance system in an extremely simple and convenient way about the desired required position.

In concrete terms determination information, which describes how the required position of the field of view relative to the marker position is to be determined, can be stored in the control system or can be determined by the system. The determination information can be fixed predetermined information, in this case, be derived from a user input and/or can especially advantageously be provided by the type or placement of the position determination unit. In particular, as will be explained in greater detail below, in addition to the marker position, orientation information describing the orientation of the position determination unit can also be determined, from which the determination information can be derived, for example, by means of an assignment specification. In a concrete, especially intuitive aspect, there can be provision for the desired location of the field of view to be determined as the middle of the field of view at least in the longitudinal direction at the marker position; thus, the position of the position determination unit/of the magnetic field sensor to be determined by the control system. In other words, by positioning the position determination unit on the patient couch or on the patient, at least in longitudinal direction, it can be defined where the middle of the field of view of the field of view of the magnetic resonance system for measurement of the magnetic resonance data is to lie. In this case, determination information thus simply specifies that the marker position, at least in relation to the longitudinal direction (z-direction), is to be moved into the middle of the field of view. This is an intuitive variant able to be understood especially clearly by a user.

Basically, however, other scenarios are also conceivable, for example, when an ability of the position determination unit to be positioned exactly at the right longitudinal position is not possible, for recording configurations to be provided in which a number of recording steps are present, and the like. In this way, for example, when a number of slice stacks with stack positions offset in the longitudinal direction are to be recorded, there is provision for the marker position to relate to the middle of the totality of the slice stacks and, thus, for the first slice stack to be moved to a position offset in the longitudinal direction, which can be specified accordingly by the determination information. A corresponding offset can already be known in the control system and the determination information can refer to additionally applying this offset.

In an especially preferred development of the present disclosure, there can be provision for the position determination unit to have an optical marker for visual description of the current marker position provided on its outer side, corresponding to the position of the magnetic field sensor in the position determination unit. For example, in particular, on all differently oriented outer surfaces of the position determination unit, a cross or the like can be provided, which specifies the position of the magnetic field sensor and thus the position that is measured as marker position. In particular, in a more extensive position determination unit, in which, as will be explained in greater detail, further components are provided and/or which are designed larger for easier handling, the user is informed directly in this manner how they can achieve an accurate positioning so that technically the desired marker position is determined by the control system.

Preferably, the position determination unit can further have an energy supply system and/or a wireless communication system, by means of which the sensor data is transferred wirelessly to the control system. The magnetic field sensor is thus connected, for example, to an energy supply device and a wireless communication system so that the position determination unit can be embodied wirelessly overall, and the ease of handling can once again be markedly improved. In particular, communication operation during the measurement of the magnetic resonance data, when the position determination unit is thus located within the magnetic resonance tunnel, is neither necessary nor activated since with the homogeneous main magnetic field in the field of view, in any event, a sensible determination of the local determination would not be possible. Thus, there can be no disturbance by the communication system in this way. The energy supply device can, for example, involve a battery, in particular, an accumulator and/or a SuperCap. There can, in particular, be provision here for the energy supply device to be charged during a measurement of alternating fields occurring in magnetic resonance data in the patient tunnel. In other words, the energy supply device can thus be embodied for energy harvesting from alternating magnetic fields, in particular, the radio-frequency and/or the gradient field, during the measurement of the magnetic resonance data. This is expedient, in particular, when, during the measurement, the position determination unit remaining on the patient couch or the patient is not located within the field of view and thus also does not disturb the measurement. If, for example, the required position is in any event only to be defined in the longitudinal direction, it can be sufficient to position the position determination unit on the edge of the patient couch, thus away from the field of view, so that, without disturbing the recording operation the energy supply device can be charged from the alternating fields employed during the measurement. In particular, when the position determination unit is arranged during the measurement of the magnetic resonance data within the field of view, techniques already proposed, which employ frequencies slightly offset relative to the Larmor frequency (magnetic resonance frequency) to transmit energy, can be applied. It is, of course, also conceivable within the framework of the present disclosure to charge the energy supply device in a conventional manner, for example, by means of a charger provided accordingly, in which the position determination unit can be arranged when it is not needed.

The communication system connects the magnetic field sensor to a corresponding wireless communication system of the magnetic resonance system, in particular, of the main magnet unit, and/or to another component of the magnetic resonance system, in particular, arranged outside the patient tunnel. For example, the further communication system can be provided in a foot, which together with the patient couch, forms a patient table. Preferably, simple known communication techniques and communication protocols for wireless communication between the position determination unit and the control system already available in the prior art can be employed here. There can be communication via Bluetooth, ZigBee, WiFi, WiGig, MedRadio, and the like, for example. In particular, medical communication techniques and communication protocols employed in any event, such as, for example, MedRadio, can thus advantageously be supplied to a further application.

Preferably, the sensor data can be transmitted on request and/or at regular intervals and/or automatically, in particular, when a number of position determination units are used, at randomly chosen time intervals. The position determination unit, when it is switched on, can thus transfer the sensor data recorded by the magnetic field sensor to the control system at regular intervals (pull) or on request (push). In particular, however, when, for example, for a multi-stage examination of a patient, a number of position determination units are used, for example, for definition of positions to be moved to in succession, it can be expedient to choose the time intervals between transfers of the sensor data at random, in particular, as regular transmission with randomly chosen time interval length and/or overall random time interval length. A random offset can also be used. In this way, the probability is minimized that the transfers of individual position determination units overlap or disturb each other.

In such cases, it should be pointed out at this juncture that preferably the raw, where necessary calibrated, sensor data is transmitted to the control system for further evaluation, however, it is entirely conceivable in exemplary aspects to carry out a pre-evaluation on the position determination unit side, in particular, by the magnetic field sensor itself. In particular, in the evaluation of the sensor data for establishing the marker position, at least one coordinate in the longitudinal direction (z-coordinate), if necessary however, also further spatial coordinates, in particular, an x-coordinate and a y-coordinate, and/or orientation information is determined, which will be discussed in greater detail below.

Preferably, a Hall sensor can be used as the magnetic field sensor and/or an orientation sensor; in particular, an acceleration sensor can be assigned to the magnetic field sensor. In this case, the magnetic field sensor especially advantageously involves a three-dimensional Hall sensor, which can provide a three-dimensional magnetic field vector at its position. However, it is also basically conceivable, in particular, when, due to the course of the stray field of the basic magnetic field, no unique assignment due to a main magnetic field map is possible, additionally to assign to the magnetic field sensor an orientation sensor, for example, an acceleration sensor or inclination sensor.

An expedient development of the present disclosure makes provision that, when a threshold value indicating an entry into the stray field of the magnetic resonance system, for the measured magnetic field, is exceeded, in particular, the magnetic field strength, of the magnetic field sensor, in particular, operated in a standby mode and/or due to measurement data of a measurement sensor of the position determination unit indicating a movement, for the entire position determination unit to be activated automatically, in particular, the magnetic field sensor switched into a full operating mode and for the communication system to be activated. In this way, the position determination unit does not have to be provided with an on switch or the like or activated externally via a switch-on signal, but it can be determined within the position determination unit itself whether, so to speak, it is to be brought into an operating position, which means positioned on the patient couch and/or the patient. For example, the magnetic field sensor can thus activate the position determination unit and switch it on as soon as it is brought into the stray field of the main magnetic field, which can be described by a suitable threshold value for the magnetic field strength. The magnetic field sensor here can initially be in an energy-saving standby mode, in which, in particular, there is merely supervision as to whether the measured magnetic field strength exceeds the threshold values. If it is, the magnetic field sensor is switched into a full operating mode, and further components, in particular, the communication system, are activated so that the sensor data can be transmitted to the control system. In addition or as an alternative, the position determination unit can also comprise a movement sensor so that the position determination unit, in particular, with sufficiently strong movement, can be brought into an active state, in particular, switched on. Preferably, when triggered by a movement sensor, the position determination unit, in particular, also its communication system, also continues to operate for a specific period of time, for example, 1 to 10 minutes, if no further strong movements follow, since where necessary no adjustments take place before the measurement of the magnetic resonance data is then actually started.

In an especially preferred development, there can be provision for orientation information describing the orientation of the position determination unit also to be determined by the control system using the sensor data of the magnetic field sensor and/or of the orientation sensor and for the control system to determine from the orientation information by means of an orientation to control information, which relates to a subsequent movement of the patient couch and/or a subsequent measurement of magnetic resonance data, and/or determination information, which describes the determination of the required position from the marker position, to determine assignment specifications, control information and/or determination information, wherein the control system controls the magnetic resonance system in accordance with the control information and/or determines the required position in accordance with the determination information. In other words, the user can use the position determination unit in order to notify the control system about further information, in that the orientations of the position determination unit are assigned semantic means by means of an assignment specification, and corresponding orientation information describing the orientation is determined. If, for example, the position determination unit has a housing providing a number of support surfaces, for example, a rectangular cube-shaped housing, laying this housing down with a first side upwards or a first side downwards can have a different meaning from laying it down with a second side facing upwards or downwards. The corresponding assignment of control information and/or determination information for orientations here can, at least in part, for set as fixed information and/or be able to be defined at least in part by a user. If, for a rectangular cube-shaped housing, as well as the six sides, an orientation in 90° degree steps is also assumed as well, 24 meanings can even be encoded. As well as the rectangular cube shape, other forms are, of course also possible, for example, dodecahedral forms and the like. In this connection, it is especially advantageous when the position determination unit comprises optical markings provided on its outer side for visual differentiation and/or assignment of the orientations. These can be provided, in particular, in addition to the optical markers indicating the position of the magnetic field sensor within the position determination unit. If, for example, with a rectangular cube-shaped position determination unit, not only the sides that point upwards are relevant, but the orientations of the sides in the horizontals are also different, it is conceivable to mark the entire side/surface with a position of the magnetic field sensor at the “x” marking the crossing point, whereby the “x” simultaneously defines part surfaces, in which distinguishable visual indicators, i.e., optical markings, can be provided for the orientations. But also, when it is only a matter of the sides themselves, markings, where necessary, in addition to the optical markers, can be provided. The optical markings can be abstract here. However, it is also possible to use semantic optical markings, for example, icons, which indicate a corresponding means assigned by means of the assignment specification.

In concrete terms, the control information can relate to a support of the patient and/or speed of movement of the patient couch and/or a local coil arrangement to be used and/or a recording program to be used, and/or a magnetic resonance sequence to be used. By means of the orientation, for example, a speed of entry of the patient couch into the patient tunnel can thus be set, for example, as slow, fast, or normal. The magnetic resonance system can also be supplied with information about the orientation of the patient, for example, “patient is lying head-first or feet-first.” Information with respect to the measurement of the magnetic resonance data itself can also be forwarded or set in this way, for example, an organ to be recorded or type of the measurement (diffusion imaging, normal imaging, etc.). Also, the information as to which local coil arrangement or local coil of a local coil arrangement is to be used can be relevant and forwarded as control information by means of the orientation.

Expediently, the activation of the patient couch by the control system can only be triggered on fulfillment, in particular, of a trigger condition, indicating a confirmation by a user. This means that the movement of the patient couch and, thus, of the patient into the patient tunnel is only initiated if a trigger event occurs, the presence of which is checked by the trigger condition. In this way, safety is enhanced, in particular, when the trigger condition indicates a confirmation by a user. What can be provided in concrete terms here is that the trigger condition evaluates the confirmation of an, in particular, redundantly embodied operating element on the main magnet unit and/or at the position determination unit. In this case, a conventional operating element, provided, for example, on the main magnet unit as a touchscreen and/or knob, can be used. Expediently, an operating element can, however, also be attached to the position determination unit so that, for example, simply only the position determination unit must be positioned, and then the movement of the patient couch into the patient tunnel can still be started immediately at the position determination unit. In this case, the operating element of the position determination unit can, for example, comprise a button and/or a touch sensor and/or a sensor measuring non-contact gestures. In particular, when a touch sensor is used and/or a sensor measuring non-contact gestures, there can also be provision in this context for the actuation to comprise a gesture, in particular, a swipe. For example, a swipe movement via the position determination unit can thus be understood as a trigger command for the movement, and the patient couch is correspondingly moved so that the desired relative position of the field of view in relation to the position determination unit, in concrete terms the magnetic field sensor, is produced. Generally speaking, this trigger function can also be designed redundantly in order to minimize malfunctions. There can be provision here, for example, for the redundant design to comprise two switches assigned to an operating element, wherein the trigger condition is then only fulfilled when both switches are determined as being actuated.

In a development of the disclosure, there can be provision for a number of position determination units, in particular, specifying a series and/or sending assigned identification information to the control system, to be used for a number of consecutive measurements of magnetic resonance data in different examination regions. Thus, a number of position determination units can also be used as position tags, in particular, distributed in the longitudinal direction, in order to indicate a number of different examination regions. Expediently, the position determination unit then possesses uniquely assigned identification information so that the control system can distinguish between them. The position determination unit can, for example, be numbered, and also be able to be seen from the outside by a user so that the examination regions can be moved to in the corresponding order of the position determination unit. As already explained in greater detail with respect to the communication, a simple minimization of a probability of disturbance during communication can be achieved by the transmission processes taking place based on random times. This avoids a more complicated design of the position determination unit in that a simple, random-based time-division multiplexing is introduced instead of a more complex-to-implement frequency-division multiplexing or the like.

As already mentioned, the position determination unit can comprise an, in particular, rectangular cube-shaped, housing, in which the magnetic field sensor is accommodated. The housing can thus also accommodate the communication system and the energy supply device. The housing can be made of plastic, for example, but also comprise a material having a certain flexibility, as is also employed, for example, in the medical field for bandages and the like.

The position determination unit can, for example, have a maximum extent of 1 to 10 cm and/or a volume of 1 to 100 cm³. Magnetic field sensors, for example, three-dimensional Hall sensors the size of chips can now be provided so that a very small design of the position determination unit is possible. For example, a rectangular cube-shaped aspect with 2 cm×2 cm×10 cm can be possible. Also conceivable, however, are cuboid aspects, for example, with a side length of 3 cm to 4.5 cm.

Preferably, the position determination unit can also have a fastener and be fastened by means of the fastener to the patient couch and/or an item of clothing and/or a cover and/or a local coil arrangement. For example, the fastener can be embodied as a clip and/or a clamp. If the position determination unit is in any event only to define the longitudinal position, the fastener can be embodied, for example, as a latch, in particular, clip, in such a way that the position determination unit can be fastened to the side of the patient couch, for example, in a slot of the patient couch running laterally in the longitudinal direction. It is also possible for the position determination unit to be able to be fastened to the patient, in particular, to clothing of the patient, and/or to local coil arrangements. Here, in particular, the use of Velcro-type materials can be useful since, for example, when a hook material is used on the fastening material side of the position determination unit, there can be interaction with a plurality of substances having, for example, eyes, in particular, also items of clothing. Flexible material, which is used in local coil arrangements that are laid on the patient, can also be suitable for bearing Velcro-type connections. For example, a housing of the position determination unit can thus be embodied as a Velcro-type cube or generally as a rectangular Velcro-type cuboid.

Along with the method, the disclosure also relates to a position determination unit for an inventive method. All of what has been stated above in relation to the inventive method is able to be transferred by analogy to such an inventive position determination unit.

In particular, the position determination unit has the magnetic field sensor, the energy supply device, and the communication system, preferably in a where necessary also at least partly flexible housing. It preferably serves no purpose in the recording of the magnetic resonance data itself but serves merely as a positioning tag, with which a user, in an especially simple manner, can define a marker position and thereby a required position of the patient on the patient couch relative to the field of view. For example, the position determination unit can have a maximum extent of 1 to 10 cm or a volume of 1 to 100 cm³. It can moreover comprise a fastener, with which it is fastened to the patient couch and/or to an item of clothing and/or a cover and/or a local coil arrangement. Corresponding optical markers and optical markings can also be arranged on the surfaces in order to indicate clearly where within the position determination unit the magnetic field sensor is arranged and/or which orientation, where necessary, indicating control information and/or determination information is being used.

Finally, the disclosure also relates to a magnetic resonance system, having a main magnet unit with an, in particular, cylindrical patient tunnel and a main magnet generating a main magnetic field, a patient couch able to be moved into the patient tunnel in a longitudinal direction, a control system and a position determination unit with a magnetic field sensor and a communication system for transmission of sensor data of the magnetic field sensor to the control system able to be freely positioned on the patient couch and/or on a patient supported on the patient couch at a marker position, wherein the control system has:
an evaluation unit for evaluation of the sensor data of the magnetic field sensor for establishing the marker position in a coordinate system of the magnetic resonance system, in which a field of view position of a field of view of the magnetic resonance system is also known,
a determination unit for establishing a desired required position of the field of view relative to the marker position from the marker position, at least in the longitudinal direction of the patient couch for a subsequent measurement of magnetic resonance data, and
a control unit for controlling the patient couch while using the marker position in such a way that the desired required position of the field of view relative to the marker position is assumed.

What has previously been stated in relation to the inventive method and the position determination unit also continues to apply for the magnetic resonance system.

### BRIEF DESCRIPTION OF THE DRAWINGS

Further advantages and details of the present disclosure emerge from the exemplary aspects described below and also with the aid of the drawings. In the figures:

FIG. 1 shows a basic diagram of an inventive magnetic resonance system;

FIG. 2 shows the functional layout of a position determination unit;

FIG. 3 shows an overhead view of the position determination unit;

FIG. 4 shows an overhead view of a further exemplary aspect of a position determination unit; and

FIG. 5 shows a flow diagram of an exemplary aspect of the inventive method.

### DETAILED DESCRIPTION

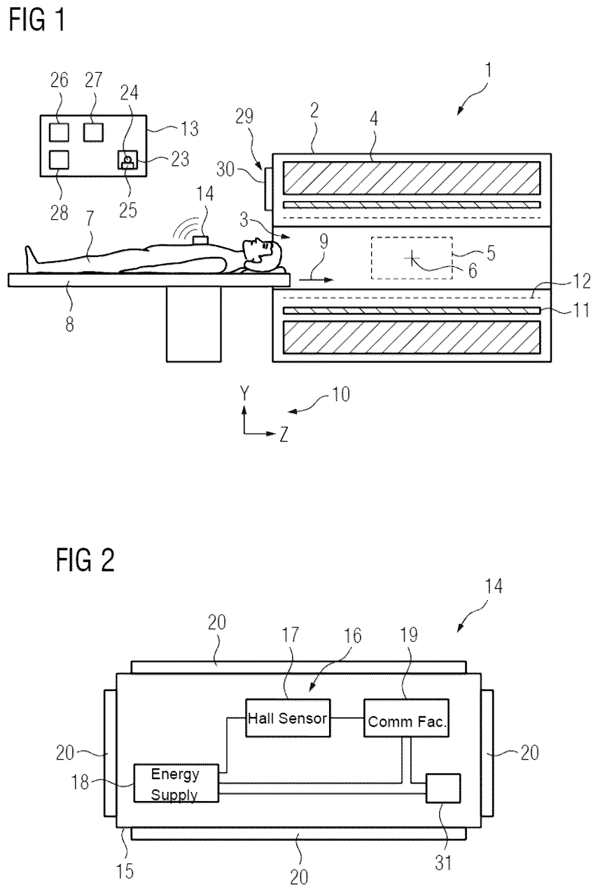

FIG. 1 is a basic diagram of an inventive magnetic resonance system 1. The magnetic resonance system 1, as is basically known, has a main magnet unit 2, which is defined in the present example as a cylindrical patient tunnel 3. The main magnet unit 2 here has a superconducting main magnet 4, which generates a main magnetic field of the magnetic resonance system 1. Within the patient tunnel 3, the main magnetic field forms the so-called homogeneity volume as field of view 5 with the middle of the field of view 6, which in the present example also forms the middle of the patient tunnel 3.

A patient 7 to be examined by magnetic resonance imaging, i.e., measurement of magnetic resonance data, can be moved into the patient tunnel 3 by means of a patient couch 8, which is shown in FIG. 1 outside the patient tunnel 3, in accordance with the arrow 9 in its longitudinal direction as the direction of movement. The longitudinal direction of the patient couch 8 also corresponds, in this case, to the longitudinal direction of the cylindrical patient tunnel 3 and is usually designated in the coordinate system 10 of the magnetic resonance system 1 as the z-direction. Also to be seen is the vertical y-direction at right angles to the z-direction; the x-direction runs at right angles to the plane of the image and is thus the direction horizontal to the z-direction.

To make it possible to record an image, the present example also has gradient coils of a gradient coil arrangement 11 adjoining the main magnet 4 in the direction of the patient tunnel 3. Optionally, a full-body radio-frequency coil arrangement 12 is also provided.

Naturally, local coil arrangements not shown in any greater detail here can also be used for examinations of the patient 7, which are permanently installed or able to be arranged on the patient couch and/or can also be laid on the patient.

The operation of the magnetic resonance system 1 is controlled by a control system 13, which is only indicated schematically here and in part of its functional layout.

In order to define a longitudinal position, i.e., position in the z-direction, which is to be positioned in the field of view 5, in concrete terms in the middle of the field of view 6, and thus to define an examination area of the patient 7 as simply as possible, a position determination unit 14 is provided, which can freely be positioned on the patient 7 and/or the patient couch 8. In this case, a positioning on the patient 7 in the present example also means a positioning on an object located on the patient 7, for example, a local coil arrangement, a cover covering the patient, and the like.

FIG. 2 shows the functional layout of the position determination unit 14 in greater detail. It can be seen that this, in a housing 15, in the middle in the current example, has a magnetic field sensor 16, which is embodied as a three-dimensional Hall sensor 17. In the present example, the three-dimensional Hall sensor 17 measures the three-dimensional magnetic field vector. Moreover, an energy supply device 18 and a communication system 19 are provided in the housing, by means of which the sensor data of the magnetic field sensor 16 can be transmitted wirelessly to the control system 13. In this case, for example, communication standards such as Bluetooth, ZigBee, WiFi, WiGig, MedRadio, and the like can be employed. The energy supply device comprises, in particular, a battery, for example, an accumulator and/or a SuperCap. In exemplary aspects, it can be embodied for energy harvesting during a measurement of magnetic resonance data, for example, from radio-frequency fields and/or gradient fields, which occur during the measurement, however, is ideally not arranged directly in the field of view 5, but, for example, to the side thereof. It can also be charged, however, by means of a charging case or the like.

The position determination unit 14 in the present example is also embodied so that it switches on automatically when it is put into use. To this end, in an inactive, switched-off state, the magnetic field sensor 16 is only active in a standby mode and recognizes when it is being brought into the stray field of the main magnetic field by a specific threshold value for the magnetic field strength being exceeded. If such an entry into this stray field of the main magnetic field is detected, the magnetic field sensor 16 switches into a full operating mode and also activates the other components, here, in particular, the communication system 19.

The communication system 19 can basically transmit sensor data on request but preferably transmits this regularly, however, for example, at regular times or at times chosen at random within an allowed interval. The variant in which the times between transmissions are chosen in accordance with a random number determined once or a random number merely determined for each time interval is useful, in particular, when a number of similarly embodied position determination unit 14 are to be employed in a simple manner without disruptive radio traffic, since then the probability of mutual disruption is minimized without any great effort having to be made.

Unique identification information of the position determination unit 14 is also transferred with the sensor data so that when a number of position determination unit 14 is used, these can be assigned. The identification information represents the only necessary difference between the position determination unit 14. A number of position determination units 14 can be employed, for example, to mark a number of examination regions to be recorded consecutively, at least in the longitudinal direction; in the control system 13, an order can then already be stored with the aid of the identification information, which can be recognizable to the outside on the housing 15.

The housing 15 can be made of a plastic and/or a woven material and/or be at least partly flexible, in particular, when the patient 7 is to be worked on. It has a fastener 20 on its outer side, which, for example, can comprise Velcro, but also a clip or other latch, for example, for latching into a slot on the side of the patient couch 8.

The housing 15 in the present example is in the shape of a rectangular cuboid and can, for example, be embodied flat, for example, with the dimensions 2 cm×2 cm×10 cm. A cuboid aspect is also conceivable, for example, with an edge length of 3 cm to 4.5 cm.

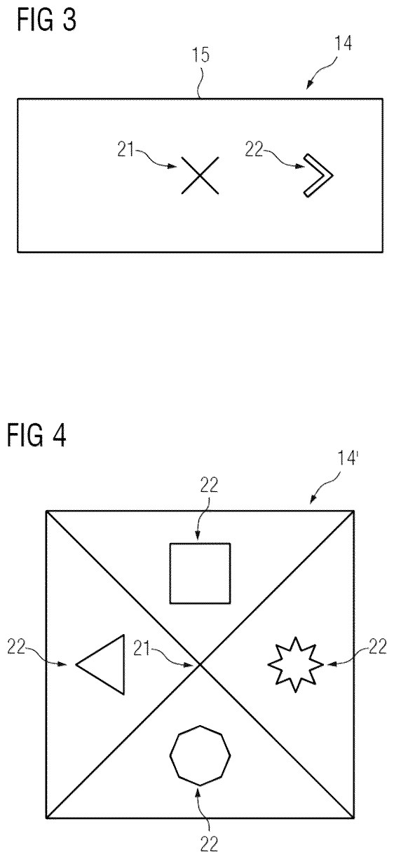

As shown in FIG. 3, visualizations are attached to the housing 15, which simplify the use of the position determination unit 14. Thus, an optical marker 21, here a cross, indicates where within the housing 15 the magnetic field sensor 16 is located. In the present example, however, an orientation of the position determination unit 16 is also relevant, through which a user can communicate further information to the control system 13, an optical marking 22 is also present. In the exemplary aspect of FIG. 3 it is only relevant which side of the position determination unit 14 is pointing upwards. Each of the sides can then be assigned control information or determination information, as will be explained in greater detail below. The symbol shown here by way of example shows a single arrow and stands for a slow movement of the patient couch 8, wherein, for example, two arrows can stand for a normal movement and three arrows for a fast speed of movement. This is to be understood by way of example; however, control information can also relate, in particular, to a support of the patient and/or to a recording program to be used and/or to a local coil arrangement to be used and/or to a magnetic resonance sequence to be used.

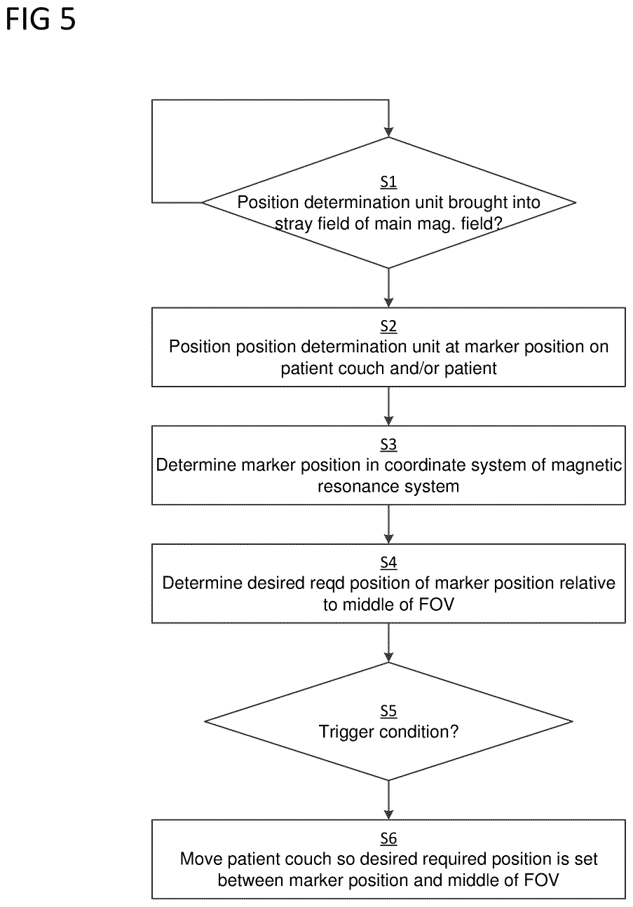

FIG. 4 shows an illustration of an overhead view of a position determination unit 14′, in which even the alignment of the corresponding surface lying at the top is relevant, and in 90° steps. Here, the optical marker 21, which shows a central location of the magnetic field sensor 16, divides the visible surface in any event into four sections, in which a corresponding optical marking 22 is provided in each case.

Returning to FIG. 1, this figure also shows schematically the functional layout of the control system 13. First of all, this has a memory unit 23, in which, in the present example, the main magnetic field map 24, which, in particular, describes the spatial course of the stray fields of the main magnetic field that are in front of the patient tunnel 3, where the patient couch 8 is located in FIG. 1 and does this in the magnetic resonance coordinate system 10. An assignment specification 25 is further also stored there, which assigns control information and/or determination information to various orientations of the position determination unit 14.

In an evaluation unit 26 of the control system 13, the sensor data of the magnetic field sensor 16, which has been transferred via the communication system 19, is evaluated in order not only to determine the current marker position of the magnetic field sensor 16 in the position determination unit 14 at least in the longitudinal direction but also orientation information describing the orientation of the position determination unit 14. The orientation information is used in order, by means of the assignment specification 25, in particular, a Look-Up table, to determine the associated control information and/or the associated determination information. Control information, which, for example, describes the speed at which the patient couch 8 is to be moved, is then applied accordingly in the further course of events.

The determination information, be it predetermined as fixed information, predetermined on the user side, or derived from the orientation information, describes which relative required position is to be determined between the marker position, i.e., the magnetic field sensor 16 or the position determination unit 14, and the field of view 5, in particular, the middle of the field of view 6. In a simple, preferred setting and one that is chosen as the prior choice, the marker position is brought by movement of the patient couch 8 at least in the longitudinal direction into line with the middle of the field of view 6. This means that what is to be positioned in the middle of the field of view 6 is directly marked by the position determination unit 14. Cases that differ from this are also conceivable, however, for example, in a multi-step recording of neighboring slice stacks, where a movement for the first slice stack can be taken into consideration when the position determination unit 14 is to mark the midpoint of all slice stacks.

By using the determination information, this required position of the field of view 5 relative to the marker position, at least in the longitudinal direction of the patient couch 8 is determined in a determination unit 27.

A control unit 28 checks for the presence of a trigger condition, which indicates that the patient couch is now to be moved so that the desired relative required position is produced. Then, in particular, during consideration of the longitudinal position, when the marker position is to be placed in the middle of the field of view 6, the patient couch 8 is moved by the longitudinal distance from the position of the middle of the field of view 6, which is known and, in particular, forms the origin of the coordinate system 10 and by the measured marker position.

The trigger condition can check, for example, in this case, whether a particular redundantly embodied operating element has been actuated. This can involve an operating element 29 on the main magnet unit 2, for example, a touchscreen 30, such as basically known in the prior art.

It is also possible, however, if necessary, additionally, as shown in the schematic function diagram of FIG. 2, to provide the position determination unit 14 with its own operating element 31, which can be a knob or a touch sensor, but which is formed in the present example by a non-contact sensor detecting gestures. If, by means of the operating element 31, a swiping movement over the position determination unit 14 is determined, the trigger condition is fulfilled, and the movement can begin. To establish the redundancy, such a sensor can be provided multiple times as an operating element 31; with a knob to be actuated, two switches can also be closed by the knob in order to provide the redundancy.

FIG. 5 summarizes the steps of a disclosed method once again in a flow diagram.

In step S1, with the position determination unit 14 switched off, the magnetic field sensor 16 in standby mode monitors whether the position determination unit 14 is being brought into the stray field of the main magnetic field. If this is the case, the position determination unit 14 is switched on. In step S2, the position determination unit 14 is positioned at a marker position on the patient couch 8 and/or the patient 7. In step S3, the marker position in the coordinate system 10 of the magnetic resonance system 1 is determined by the evaluation unit 26 from the sensor data of the magnetic field sensor 16 by reconciliation with the main magnetic field map 24.

In step S4 a desired required position of the marker position relative to the middle of the field of view 6 is determined by the determination unit 27. Then, in the control unit 28 in step S5, a check is made for the presence of the trigger condition as to whether, for example, a user has swiped over the position determination unit 14. If the condition is fulfilled, in step S6 the patient couch 8 can be moved so that the desired required position is set between the marker position and the middle of the field of view 6, thus, in particular, is positioned in the location defined by the position determination unit 14 in the middle of the field of view 6.

Naturally, control information is also taken into consideration if this has been specified accordingly by means of the assignment specification 25 during the operation of the patient couch 8 and/or the subsequent measurement of magnetic resonance data.

It should be pointed out once again at this juncture that, instead of or in addition to the standby mode of the magnetic field sensor 16 and the checking against at least one threshold value, a movement sensor can also be used, which describes the movement and use of the position determination unit 14.

Although the disclosed aspects have been illustrated and described in more detail by the preferred exemplary aspect, the disclosure is not restricted by the disclosed examples, and other variations can be derived herefrom by the person skilled in the art without departing from the scope of protection of the disclosure.

1. A method for positioning a patient couch in a patient tunnel of a magnetic resonance system, wherein the patient tunnel is defined by a main magnet unit with a main magnet operable to generate a main magnetic field, the method comprising:
positioning a position determination unit comprising a magnetic field sensor at a marker position on the patient couch or on a patient supported on the patient couch;
evaluating sensor data of the magnetic field sensor using a control system to establish the marker position in a coordinate system of the magnetic resonance system, in which a field of view position of a field of view of the magnetic resonance system is known;
determining a desired required position of the field of view relative to the marker position for a subsequent measurement of magnetic resonance data by the control system from the marker position at least in a longitudinal direction of the patient couch corresponding to a direction of movement of the patient couch; and
controlling the patient couch by the control system, using the marker position and the field of view position, in such a way that the desired required position of the field of view relative to the marker position is set.

2. The method as claimed in claim 1, further comprising:
determining a desired location of the field of view as a middle of the field of view at least in the longitudinal direction at the marker position by the control system.

3. The method as claimed in claim 1, wherein the position determination unit comprises an optical marker on its outer side, corresponding to the position of the magnetic field sensor in the position determination unit, for visual description of a current marker position.

4. The method as claimed in claim 1, wherein the position determination unit further comprises an energy supply device and/or a wireless communication device operable to transfer the sensor data wirelessly to the control system.

5. The method as claimed in claim 4, wherein the energy supply device is charged by alternating fields occurring during a measurement of magnetic resonance data in the patient tunnel and/or the sensor data is transmitted automatically on request and/or at regular intervals.

6. The method as claimed in claim 1, wherein the magnetic field sensor is a Hall sensor and/or is an orientation sensor.

7. The method as claimed in claim 6, wherein by the control system, using the sensor data of the magnetic field sensor and/or of the orientation sensor, orientation information describing the orientation of the position determination unit is determined and the control system, from the orientation information, using an orientation to control information, which relates to a subsequent movement of the patient couch and/or a subsequent measurement of magnetic resonance data, and/or determination information, which describes the determination of the required position from the marker position, assignment specification to be assigned, control information and/or determination information, wherein the control system controls the magnetic resonance system in accordance with the control information and/or determines the required position in accordance with the determination information.

8. The method as claimed in claim 7, wherein the control information relates to a support of the patient and/or to a speed of movement of the patient couch and/or to a local coil arrangement to be used and/or to a recording program to be used and/or to a magnetic resonance sequence to be used, and/or that the position determination unit comprises optical markings for visual distinction, and/or assignment of the orientations provided on its outer side.

9. The method as claimed in claim 1, wherein activation of the patient couch by the control system only takes place on fulfillment of a trigger condition.

10. The method as claimed in claim 9, wherein the trigger condition evaluates actuation of an operating element on the main magnet unit and/or on the position determination unit.

11. The method as claimed in claim 1, wherein identification information specifying a number and/or an assigned position determination unit sending to the control system, is used for a number of consecutive measurements of magnetic resonance data in different examination regions.

12. The method as claimed in claim 1, wherein the position determination unit has a housing in which the magnetic field sensor is accommodated, and/or has a maximum extent of 1 to 10 cm and/or volume of 1 to 100 cubic centimeters and/or is fastened using a fastener to the patient couch and/or to an item of clothing and/or to a cover and/or to a local coil arrangement.

13. The method as claimed in claim 12, wherein the fastener comprises a clip and/or Velcro.

14. A position determination device configured to perform the method as claimed in claim 1.

15. A magnetic resonance system having a main magnet unit with a patient tunnel and a main magnet operable to generate a main magnetic field, a patient couch movable into the patient tunnel in a longitudinal direction, a control system, and a patient supported on the patient couch and/or a position determination device with a magnetic field sensor freely supported on the patient couch positionable at a marker position and a communication device to transmit sensor data of the magnetic field sensor to the control system, wherein the control system comprises:
an evaluation unit operable to evaluate the sensor data of the magnetic field sensor to establish the marker position in a coordinate system of the magnetic resonance system, in which a field of view position of a field of view of the magnetic resonance system is also known,
a determination unit operable to establish a desired required position of the field of view relative to the marker position from the marker position at least in the longitudinal direction of the patient couch for a subsequent measurement of magnetic resonance data; and
a controller operable to activate the patient couch while using the marker position in such a way that the desired relative required position of the field of view for the marker position is assumed.
